(19) United States
(12) Patent Application Publication
(10) Pub. No.: US 2011/0282233 A1
(43) Pub. Date: Nov. 17, 2011

(54) LOW-POWER REAL-TIME SEIZURE DETECTION SYSTEM

(76) Inventors: Shriram Raghunathan, Houston, TX (US); Pedro Irazoqui, West Lafayette, IN (US); Sumeet Kumar Gupta, West Lafayette, IN (US); Kaushik Roy, West Lafayette, IN (US)

(21) Appl. No.: 13/144,452

(22) PCT Filed: Jan. 13, 2010
(86) PCT No.: PCT/US10/20913

§ 371 Date: Aug. 5, 2011

Related U.S. Application Data
(60) Provisional application No. 61/144,327, filed on Jan. 13, 2009.

(51) Int. Cl. A61B 5/0476

(52) U.S. Cl. 600/544

(57) ABSTRACT

A method for detecting an approaching seizure is disclosed. The method includes receiving an analog neurosignal from an electrode, comparing the analog neurosignal with a predetermined amplitude threshold to generate a plurality of digital transitions corresponding to the analog neurosignal transitioning from a first level below the predetermined amplitude threshold to a second level above the predetermined amplitude threshold, generating a plurality of comparator signals, each comparator signal of the plurality of comparator signals corresponding to a comparison of a length of time between a pair of digital transitions of the plurality of digital transitions and a predetermined temporal threshold, and generating a seizure detection signal if at least two of the comparator signals of the plurality of comparator signals are enabled. A seizure detection system for detecting an approaching seizure is also disclosed.

### TECHNICAL FIELD

The present invention generally relates to seizure detection systems and particularly to implantable seizure detection systems.

### BACKGROUND

Epilepsy is the second most common neurological disorder affecting over 1% of the world's population. An epileptic seizure commonly manifests into physical signs such as convulsion. However, prior to a clinical onset of the seizure changes in a neuro signal of a monitored subject may be observed. Such changes are categorized as the electrographic onset of the seizure.

The electrographic onset of a seizure is evidenced by a sustained number of low amplitude and high frequency electrical bursts in the neuro signal. The neuro signal may be obtained via an electrode or electrode array transferring electrical signals from a part proximal to the seizure onset zone in the brain of the subject. Although not always readily identifiable, these low amplitude and high frequency bursts are different than other non-sustained electrical activity in the neuro signal. The electrographic onset usually does not accompany or coincide with physical signs of the seizure in the subject. Since the electrographic onset usually precedes the clinical onset, it is desirable to provide a detection warning of the approaching seizure.

Experts in the field of neuroscience broadly divide the field of seizure monitoring into prediction/early detection of an approaching seizure, and detection/identification of the seizure after the electrographic onset of the seizure. These regimes differ in the amount of warning time each regime makes available prior to the clinical onset of the seizure. In particular, seizure prediction/early detection algorithms analyze the neuro signal for characteristic changes before or during the early stages of the electrographic seizure onset in order to provide a prediction/early detection warning signal prior to the clinical onset. Conversely, seizure detection/identification algorithms may provide a detection/identification after the electrographic onset. While seizure detection/identification algorithms have received a fair amount of favorable reviews, seizure prediction/early detection has remained controversial due to uncertainty in the pre-ictal state in the brain, i.e., the state of the brain prior to the electrographic seizure onset, which is different from its inter-ictal state, i.e., the state of the brain during the seizure.

Seizure detection/identification algorithms face less challenging requirements from a hardware perspective when used in clinical bedside applications, mainly because delay and hardware feasibility are not critical considerations in these algorithms. In other words, a sophisticated computer can run a seizure detection/identification algorithm on large amounts of pre-recorded neuro signal to detect/identify a seizure without having to make the detection/identification in real time. However a prediction/early detection algorithm relies on accurately identifying either time or frequency domain components of the neurosignal immediately after the electrographic onset. While a bright demarcation line between prediction and early detection may not exist, a recent classification made by the international workshop on seizure prediction (IWSP, Kansas city, 2009) considers any detection less than ten (10) seconds prior to the electrographic onset of the seizure to be in the classification of early detection. Also, while an exact quantification of an electrographic seizure onset may remain subject to interpretation of a number of variables, e.g., amplitude and frequency components of the neurosignal, a detection warning can be helpful in alerting a seizure-prone patient to brace for the oncoming seizure or for a seizure prevention technique to effectively prevent the seizure from occurring.

### SUMMARY

A method for detecting an approaching seizure is disclosed. The method includes receiving an analog neurosignal from an electrode, comparing the analog neurosignal with a predetermined amplitude threshold to generate a plurality of digital transitions corresponding to the analog neurosignal transitioning from a first level below the predetermined amplitude threshold to a second level above the predetermined amplitude threshold, generating a plurality of comparator signals, each comparator signal of the plurality of comparator signals corresponding to a comparison of a length of time between a pair of digital transitions of the plurality of digital transitions and a predetermined temporal threshold, and generating a seizure detection signal if at least two of the comparator signals of the plurality of comparator signals are enabled.

The method further includes separating the analog neurosignal to a first signal having a first frequency range and to a second signal having a second frequency range.

The method as stated above wherein the first signal includes frequency components from 0 to 10 Hz.

The method as stated above wherein the second signal includes frequency components from 10 to 500 Hz.

The method further includes averaging the amplitude of the first signal, and updating the predetermined amplitude threshold based on the averaged amplitude of the first signal.

The method as stated above wherein the predetermined amplitude threshold is a predetermined multiple of the averaged amplitude of the first signal.

The method further includes synchronizing the plurality of digital transitions to a clock.

The method further includes counting a number of clock cycles between a pair of synchronized digital transitions to generate a count of a length of time between the pair of synchronized digital transitions, and latching the count to generate an input to a comparator to compare the latched count with a predetermined count corresponding to the predetermined temporal threshold to generate a comparator output signal of the plurality of comparator output signals.

The method as stated above wherein the predetermined amplitude threshold and the predetermined temporal threshold are adjusted according to an accumulated history of false positives and false negatives.

The method as stated above wherein the number of comparator signals of the plurality of comparator signals being enabled causing generation of the seizure detection signal is adjusted according to an accumulated history of false positives and false negatives.

A seizure detection system for detecting an approaching seizure is also disclosed. The seizure detection system includes a signal amplitude comparator configured to compare an analog neurosignal and a predetermined amplitude threshold and generate a plurality of digital transitions corresponding to the analog neurosignal transitioning from a first level below a predetermined amplitude threshold to a second level above the predetermined amplitude threshold, and at least one comparator generating a plurality of comparator signals corresponding to a comparison of a length of time between a pair of digital transitions of the plurality of digital transitions and a predetermined temporal threshold, wherein a seizure detection signal is generated if at least two of the comparator signals of the plurality of comparator signals are enabled.

The seizure detection system further includes a low pass filter configured to separate the analog neurosignal to a first signal having a first frequency range and to a second signal having a second frequency range.

The seizure detection system as described above wherein the first signal includes components from 0 to below 10 Hz.

The seizure detection system as described above wherein the second signal includes components from 10 to 500 Hz.

The seizure detection system further includes a moving average block configured to average the amplitude of the first signal, and an updating circuit configured to update the predetermined amplitude threshold based on the averaged amplitude of the first signal.

The seizure detection system as described above wherein the predetermined amplitude threshold is a predetermined multiple of the averaged amplitude of the first signal.

The seizure detection system further includes a synchronization circuit configured to synchronize the plurality of digital transitions to a clock.

The seizure detection system further includes a counter configured to count a number of clock cycles between a pair of synchronized digital transitions to generate a count of a length of time between the pair of synchronized digital transitions, and a latch configured to latch the count to generate an input to a comparator of the plurality of comparators to compare the latched count with a predetermined count corresponding to the predetermined temporal threshold to generate a digital comparator output.

The seizure detection system as described above wherein the predetermined temporal threshold is adjusted according to an accumulated history of false positives and false negatives.

The seizure detection system as described above wherein the number of comparator signals of the plurality of comparator signals being enabled causing generation of the seizure detection signal is adjusted according to an accumulated history of false positives and false negatives.

The described features and advantages, as well as others, will become more readily apparent to those of ordinary skill in the art by reference to the following description and accompanying drawings.

### BRIEF DESCRIPTION OF DRAWINGS

FIG. 1 is a block diagram of an exemplary system for data collection and processing of neurosignals including interface with external computing components;

FIG. 2 depicts a block diagram of the data collection and processing components depicted in FIG. 1;

FIG. 3 depicts a block diagram of a preconditioning block depicted in FIG. 2;

FIG. 4 depicts a block diagram of an inter-event interval (IEI) detection block depicted in FIG. 2;

FIG. 5 depicts a flowchart of a process performed by the hardware depicted in FIGS. 1-4;

FIG. 6 depicts a plot of a typical neurosignal and other related signals;

FIG. 7 depicts a magnified portion of the plot depicted in FIG. 6;

FIG. 8 depicts a typical amplitude vs. time graph of a neurosignal showing criterion for detection;

FIG. 9 depicts a graph of the relationship between a factor K and detected IEI showing a log-normal distribution;

FIG. 10 depicts a graph of measured detection delays from each of implanted animals in a study;

FIG. 11 depicts a graph of progression of three seizures over time; and

FIG. 12 depicts a graph of amplitude vs. time of a neurosignal during a seizure.

### DETAILED DESCRIPTION

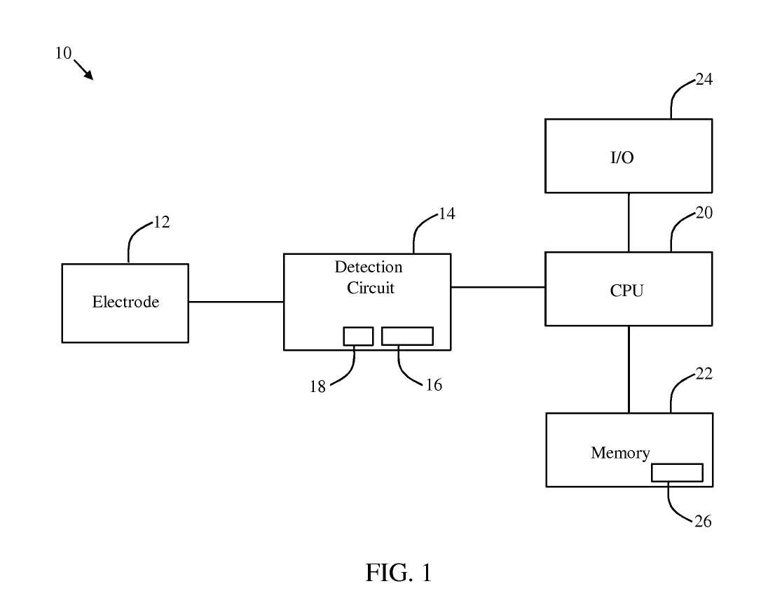

FIG. 1 depicts a representation of a seizure detection (SD) system. The SD system 10 includes an electrode 12, a detection block 14 which includes a detection circuit 16 and a memory block 18, a processing circuit 20, a memory block 22 and an input/output (I/O) device 24. The I/O device 24 may include a user interface, graphical user interface, keyboards, pointing devices, remote and/or local communication links, displays, and other devices that allow externally generated information to be provided to the SD system 10, and that allow internal information of the SD system 10 to be communicated externally.

The processing circuit 20 may suitably be a general purpose computer processing circuit such as a microprocessor and its associated circuitry. The memory 27 may suitable be various memory and data storage elements associated with a general purpose computer. Within the memory 22 are various instructions in a program instruction block 26. The processing circuit 20 is configured to execute the program instructions 26 to carry out the various operations described herein, as well as other operations.

The processing circuit is also connected to the I/O device 24 to receive data from, and present data to a user. The processing circuit 20 is also connected to the detection block 14 to receive data from, and send data to, the detection block 14. The data communicated between the processing circuit 20 and the detection block 14 includes configuration data and a seizure detection alarm signal, as well as other data. The detection block stores the configuration data that is communicated between the processing circuit 20 and the detection block 14 in the memory block 18. The memory block 18 may include random access memory (RAM), read only memory (ROM), programmable read only memory (PROM), erasable programmable read only memory (EPROM), or electrically erasable read only memory (EEPROM), and other types of memory known in the art suitable for storing data. The data may be of the type that continuously changes, or of the type that changes during programming of the detection block 14.

The detection circuit 16 interfaces with and processes data from the processing circuit 20 and the electrode 12. The electrode 12 may be a single electrode or an electrode array. The electrode 12 is a device that is connectable to animal tissue to detect neuro signals. The electrode 12 may be an implantable type or of a type that is adhereable to a skull.

In operation, clinicians interface with the processing circuit 20 via the I/O device 24 in order to provide parameters that the processing circuit communicates to the detection block 14. The clinicians also receive data from the I/O device 24 that is generated by the detection block 14. The processing circuit 20 receives the parameters from the I/O device 24 and communicates these parameters to the detection block 14. The processing circuit also communicates data form the detection block to the I/O device 24. The processing circuit 20 transfers data from the I/O device 24 to the detection block 14 and from the detection block 14 to the I/O device in accordance with the program instructions that are stored in the memory block 22. The detection block 14 receives neurosignals from the electrode 12. Based on the parameters provided by the processing circuits 20, the detection block 14 processes the neurosignals in accordance with an algorithm, described herein, and is thereby configured to generate a seizure detection signal to the processing circuit 20.

The processing circuit 20 communicates the seizure detection signal to downstream circuits in a closed loop system to take certain actions, e.g., alert a human subject of an impending seizure event. The term closed loop is intended to indicate a detection subsystem followed by a stimulation subsystem. The detection system detects an approaching seizure and provides sufficient warning to the stimulation subsystem to provide the appropriate stimulation to terminate the seizure. The stimulation subsystem may be configured to terminate progression of the seizure by locally stimulating the brain with electrical signals, chemical stimulants, and/or optical stimulation.

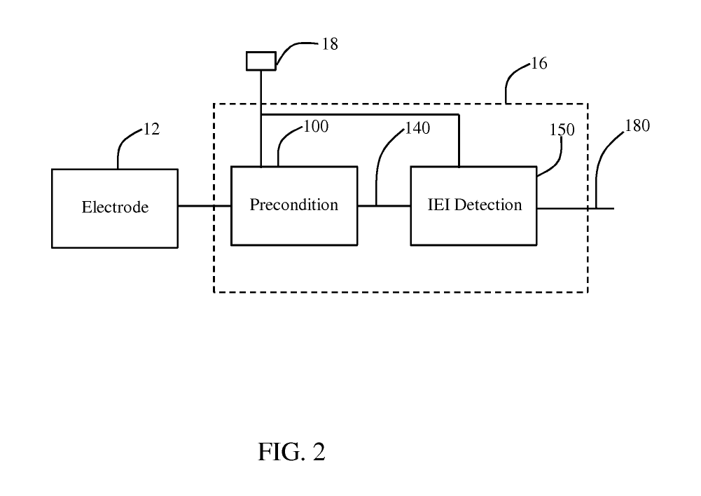

FIG. 2 depicts a block diagram of the detection circuit 16 and its interface with the electrode 12 and the memory block. The detection circuit 16 includes a preconditioning block 100 and an inter-event interval (IEI) detection block 150. The preconditioning block 100 connects to the electrode 12 and also to the memory block 18. The preconditioning block 100 also connects to the IEI detection block 150 by an interface 140. The IEI block 150 also connects to the memory block 18. The IEI detection block 150 provides an output on an interface 180.

The precondition block 100 receives raw neuro signals from the electrode 12. The preconditioning block 100 conditions the raw data obtained from the electrode 12 based on criteria that are stored in the memory block 18 and which the preconditioning block 100 receives from the memory block 18. The preconditioning block 100 then provides the conditioned data to the IEI detection block 150 over the interface 140. The IEI detection block 150 is configured to detect approaching seizures and generates a seizure detection signal on the output 180.

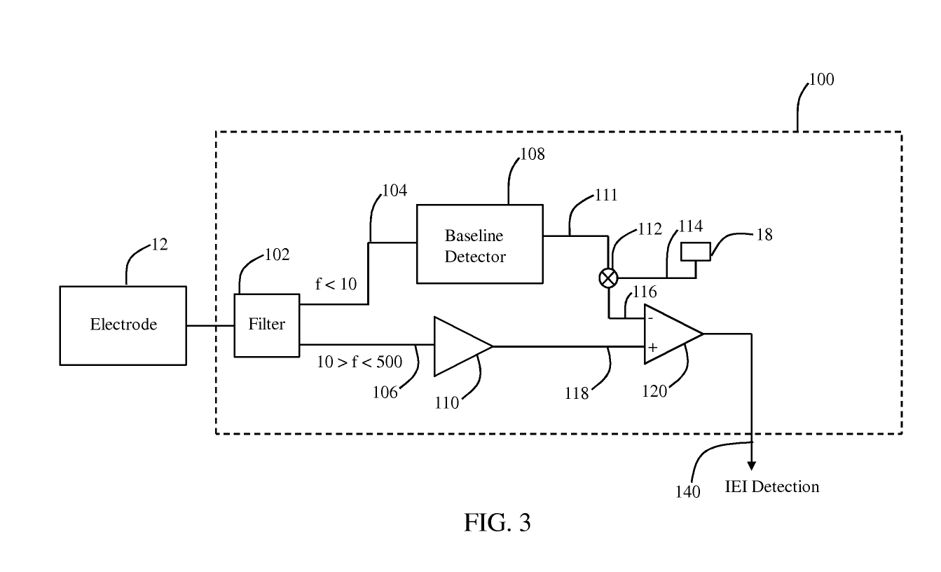

FIG. 3 depicts a schematic diagram of the preconditioning block 100. The preconditioning block 100 includes a filter 102, a baseline detector 108, an amplifier 110, a multiplier 112, and a comparator 120. The filter 102 connects to the electrode 12. The filter 102 also connects to the baseline detector 108 and to the amplifier 110 via respective interfaces 104 and 106. The baseline detector 108 connects to the multiplier 112 via an interface 111. The multiplier 112 couples to the memory block 18 via an interface 114. The multiplier 112 is operably coupled to provide an output to the comparator 120 via an interface 116. The amplifier 110 also connects to the comparator 120 via an interface 118. The comparator 120 connects to the output 140.

The filter 102 receives raw data from the electrode 12 and filters the raw data into a first signal having a first frequency range and a second signal having a second frequency range. The filter 102 places the first signal on the interface 104 and places the second signal on the interface 106. The first signal has a frequency range of 0 Hz, i.e., the DC component of the neuro signal, to about 10 Hz. The second signal has a frequency range of about 10 Hz to about 500 Hz. It is to be appreciated that the frequency ranges of the first and second signals are provided as exemplary ranges. Other frequency ranges are also possible.

The baseline detector 108 receives the first signal on the interface 104 and determines a baseline value of the neuro signal. The baseline detector 108 can include a moving average block that generates a moving average of the first signal using a window for averaging as is known in the art. In such an embodiment, the memory block 18 can provide a digital representation of the moving average window to the baseline detector 108. The baseline detector 108 would then be configured to implement the moving average window on the first signal. The baseline detector 108 calculates and places the calculated representation of the neurosignal baseline on the interface 111. The neurosignal baseline is used as an input to the multiplier 112. The multiplier 112 is configured to receive and interpret a multiplication factor “K” from the memory block 18 on the interface 114. The multiplier 112 uses the multiplication factor K to multiply the neurosignal baseline placed on the interface 111 by the factor K to generate an amplitude threshold and places the amplitude threshold on the interface 116.

Meanwhile, the amplifier 110 amplifies the second signal which is placed on the interface 106. The amplifier 110 provides the amplified version on the interface 118. The comparator 120 then compares the signals on the interfaces 116 and 118 to generate the output 140. As the amplified version of the second signal rises from a level below the amplitude threshold to a level above the amplitude threshold, the comparator output 140 rises from a digital low level to a digital high level. As the signal on interface 118 falls below from a level above the amplitude threshold to a level below the amplitude threshold, the comparator output 140 falls from a digital high level to a digital low level. In order to reduce jitter on the comparator output 140, the comparator 120 can be implemented with a sufficient level of hysteresis. While the comparator 120 is used in the preconditioning block 100, the function served by the comparator 120 can be performed by a single bit A/D block.

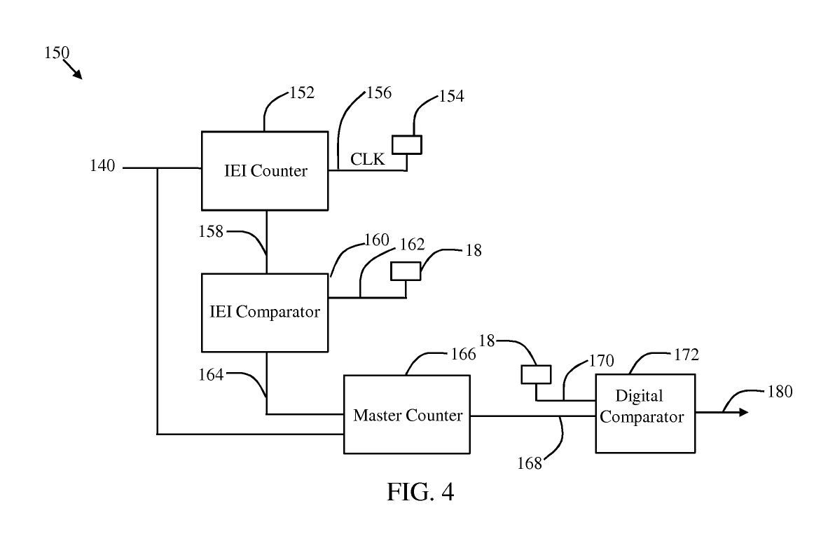

FIG. 4 depicts the IEI detection block 150. The IEI detection block 150 includes an IEI counter 152, a clock generator 154, an IEI comparator 160, a master counter 166, and a digital comparator 172. The preconditioning block 100 connects to the IEI counter 152 via the comparator output 140. The IEI counter 152 connects to the clock generator 154 via an interface 156. The IEI counter 152 also connects to the IEI comparator 160 via an interface 158. The ID comparator 160 connects to the memory block 18 via an interface 162. The IEI comparator 160 connects to the master counter 166 via an interface 164. The preconditioning block 100 also connects to the master counter 166 via the comparator output 140. The master counter 166 connects to the digital comparator 172 via an interface 168. The digital comparator also connects to the memory block 18 via an interface 170. The output of the digital comparator 172 is placed on the interface 180.

The IEI counter 152 maintains an IEI count therein. The IEI counter 152 is configured such that each rising edge of the comparator output 140 resets the IEI count to zero (0). The IEI counter 152 is further configured such that each rising edge of a clock signal placed on the interface 156 increments the IEI count. The IEI counter 152 can further be configured to operably latch the comparator output 140, e.g. by a Set-Reset (S-R) flip flop (not shown), and synchronize the latched rising edge with the clock signal 156, e.g., by a D-type flip flop (also not shown). The IEI counter 152 places the IEI count on the interface 158 in the form of a digital number.

The comparator 160 receives an IEI threshold from the memory block 18 via the interface 162. If the IEI count reaches the IEI threshold, the IEI comparator 160 places a reset signal on the interface 164. The master counter, which is a counter that maintains a master count, resets its master count to zero (0) in response to the reset signal issued by the IEI counter 160. At the same time, the IEI comparator 160 can also optionally reset the IEI count of the IEI counter 152 to zero (0). The reset signal that the IEI comparator 160 generates and places on the interface 164 can be in the form of a pulse that is latched, e.g., by an S-R flip flop (not shown).

The master counter 166 increments the master count in response to the rising edges of the comparator output 140. The master counter 166 places the master count on the interface 168 in the form of a digital number which is provided as an input to the digital comparator 172. Each increment of the master count by the master counter 166 indicates the length of time between consecutive neurosignal peaks (filtered by the filter 102, amplified by the amplifier 110, and compared with the amplitude threshold by the comparator 120) has met the IEI threshold criterion, i.e., the length of time was less than the IEI threshold (a temporal threshold).

The digital comparator 172 receives a count threshold from the memory block 18 via the interface 170. The digital counter 172 compares the master count to the count threshold. Once the master count reaches the count threshold, the digital comparator 172 generates a seizure detection alarm signal on the interface 180. The digital comparator 172 mat be configured to latch the alarm signal, e.g., by an S-R flip flop. The alarm signal indicates an approaching seizure. In addition to the alarm signal, the comparator 172 can also latch the master count and place the latched master count on the interface 180 to be communicated to downstream circuits. The master count indicates the number of consecutive neurosignal peaks that have met the IEI threshold criterion.

While the foregoing description indicated sensitivity to rising edges of the comparator output 140, it is appreciated that sensitivity to falling edges of the comparator output 140 can also be implemented. Also, in order to avoid conflict between edge transitions on the comparator output 140 and the reset signal placed on the interface 164, the reset signal (164) and can be synchronized to the clock signal inside the master counter 166, not shown.

Furthermore, while the foregoing description included one counter indicated as the IEI counter 160, which generated the IEI count, one comparator indicated as the IEI comparator 160 which compares the IEI count with the IEI threshold, one counter indicated as the master counter 166 which generates the master count, and one comparator indicated as the digital comparator 172 which compares the master count to the count threshold, in an alternative embodiment a cascaded set of counters and comparators can be implemented. In this embodiment, the output of a first stage IEI comparator (160₁) can be used to enable a second stage IEI comparator (160₂), and so on. In one form, the number of stages is fixed and equals the count threshold. In this form the output of the last stage is equivalent to the output of the digital comparator 172. In another form, a larger number of stages may be implemented. In this form, more flexibility can be provided to a clinician by choosing output of the stage that provides the most robust results, e.g., least number of false positives.

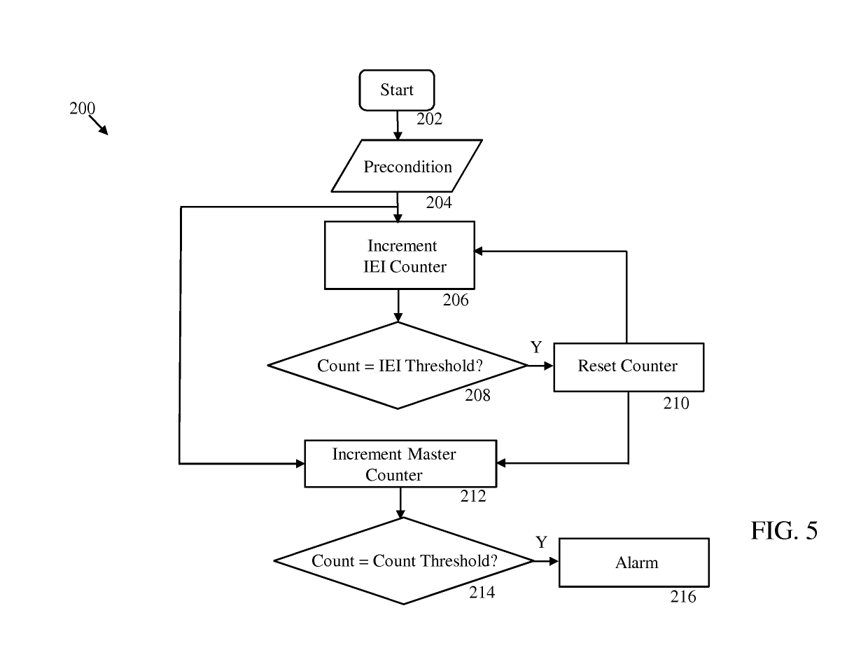

In operation and as indicated in FIG. 5, the preconditioning block 100 receives raw neurosignal (block 202). The preconditioning block 100 preconditions the neurosignal and generates a digital comparator output 140 (block 204). The IEI counter 152 resets the IEI count to zero (0) in response to a digital transition of the comparator output 140 and increments the IEI count in response to the clock signal (block 206). The IEI comparator 160 compares the IEI count to the ID threshold (block 208). If the IEI count equals the IEI threshold then the IEI comparator reset the master count on the master counter 166 and optionally resets the IEI count on the IEI counter 152 (block 210). Meanwhile, the master counter 166 increments the master count in response to the digital transitions of the comparator output 140 (block 212). The digital comparator 172 compares the master count with the count threshold (block 214). If the master count equals the count threshold, then the digital comparator 172 generates a seizure detection alarm signal (block 216).

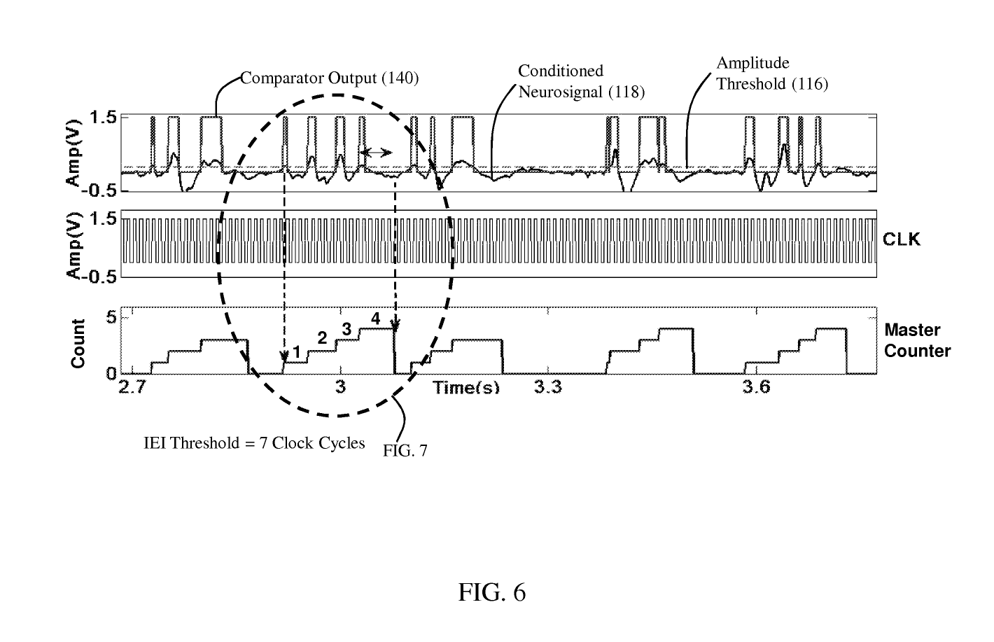

FIG. 6 depicts a plot of a neuro signal and other related signals. In the top trace, the comparator output 140 is shown over a conditioned neurosignal (interface 118). The comparator output 140 is a digital signal that transitions from a digital low to a digital high in response to the conditioned neurosignal crossing the amplitude threshold (interface 116). In the middle trace, the clock signal (interface 156) is depicted. In the bottom trace the master count (interface 168) is depicted.

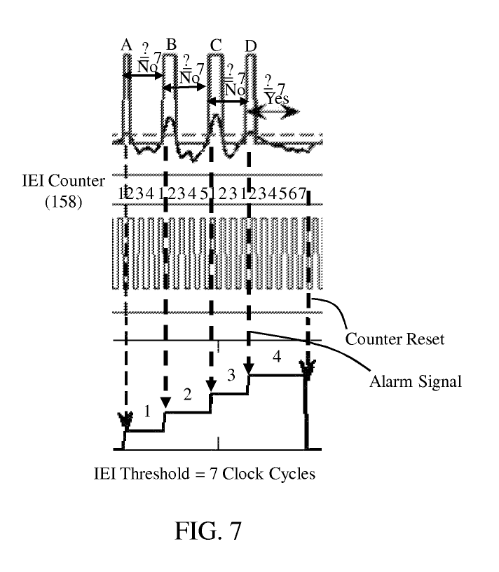

FIG. 7 depicts an enlarged portion of a section of FIG. 6. The IEI counter 152 resets the IEI count (interface 158) to zero with each rising edge of the comparator output 140. The IEI counter 152 increments the IEI count with each rising edge of the clock signal (interface 156), until the next rising edge of the comparator output 140, at which point the IEI counter 152 resets again. Therefore, the IEI counter 152 has a count of 4 for the spacing between the rising edges marked as A and B, a count of 5 for the rising edges marked as B and C, and a count of 3 for the rising edges marked as C and D. In the plots of FIGS. 6 and 7, the IEI threshold is 7. Since there is not another rising edge of the comparator output within 7 counts of the rising edge marked as D, the master counter is reset on the seventh rising edge of the clock after the rising edge marked as D.

Meanwhile with each rising edge of the comparator output 140, the master counter 166 counts up by one. Therefore, with the rising edge marked as A, the master counter 166 increments the master count from 0 to 1. Similarly, for the rising edge marked as B, the master counter 166 increments the master count from 1 to 2, for the rising edge marked as C, the master counter 166 increments the master count from 2 to 3, and for the rising edge marked as D the master counter 166 increments the master counter from 3 to 4. Once the IEI counter 152 generates the reset signal, however, the master counter resets the master count back to 0. If the count threshold in the plots of FIGS. 6 and 7 was set to 4, as soon as the master counter reaches the count of 4 (at about the rising edge marked as D), the digital comparator 172 would generate an alarm signal in the form of a pulse or a signal going from a digital low to a digital high.

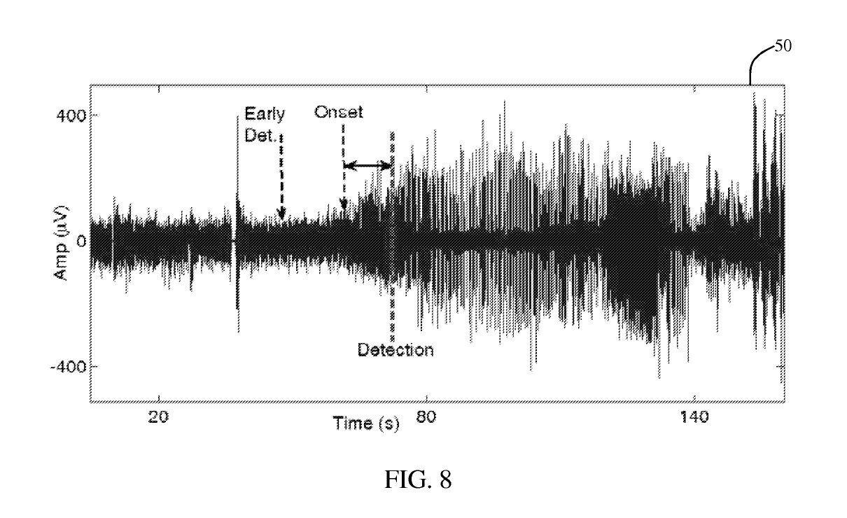

The progression of a seizure has been commonly documented to follow a relatively low-amplitude high-frequency start (tonic), followed by a higher-amplitude low-frequency middle period (clonic) and concluded with a significant decrease in the amplitude of the neurosignal. In general, it is observed that there is a decreased randomness during the ictal period and an increased overall amplitude of signals compared to non-seizing parts. The methods and apparatuses discussed hereinabove provide a capability to measure local field potential data sampled at 1525 Hz from microelectrodes (reference numeral 12) to detect the high-frequency onset of an electrographic seizure at its epileptogenic focus. An electrographic onset (EO) at the focus usually precedes a clinical onset (CO) of the seizure, allowing for the detection method and apparatus described hereinabove to be employed. FIG. 8 depicts an amplitude vs. time graph of a neurosignal showing the time points that demarcate an early detection signal before the EO and a delayed detection signal after the EO according to the guideline presented by the international workshop on seizure prediction (IWSP). The EO occurs at about 60 seconds marked as “onset.” Detection in the range between about 48 seconds to about 61 seconds is categorized by IWSP as early detection. Detections earlier than 48 seconds is categorized by IWSP as prediction. Detection based on the algorithm and the system described hereinabove is made at about 72 seconds (marked as “Detection”).

In order to examine the efficacy of the method and apparatus described hereinabove, a total of ten female long Evans rats (250 to 350 g) were used in a study. Seizure data from six of the ten animals are used in the study after accounting for the loss of animals due to damaged head caps, post surgical and kainate treatment complications. All procedures described below remained consistent for each animal. Anesthesia was induced via 5% isoflurane in 2 L miⁿ⁻¹_{O2} and maintained using 0.5-3% isoflurane in 2 L miⁿ⁻¹_{O2}. Post induction, the surgical site was shaved and cleaned with alternating scrubs of dial surgical scrub and betadine. Using a standard stereotactic frame, e.g., David Kopf Instruments, Tujunga, Calif., USA, a midline incision was made and the skull was cleaned to expose lambda, bregma and a proposed craniotomy site. Three bone screw locations and the proposed craniotomy site were marked prior to drilling with a sterile ruler and cauterizer. To locate and access the dentate gyrus, a 1 m^{m2} craniotomy was made 3.5 mm posterior and 2.0 mm lateral to bregma via stereotaxis. Prior to insertion, the electrode pair was mounted on a sterile micromanipulator and re-sterilized in a 70% ethanol in dH2O solution. A twisted-pair two-channel stainless steel electrode (Plastics One, Roanoke, Va., USA) was inserted at ˜100-300 mm miⁿ⁻¹ such that the exposed tips were 3.5 mm ventral to the cortex in the dentate gyrus. The electrode assembly consists of two 4 mm long polyimide-insulated stainless steel electrodes (0.280 mm diameter with insulation) in a twisted pair configuration with a separate uninsulated, stainless steel ground/reference wire. Kwik-Cast silicone elastomer (World Precision Instruments, Inc., Sarasota, Fla., USA) was used to cover the remaining exposed cortex followed by a liberal application of standard dental cement to cover the remaining exposed, pre-cleaned skull surface.

Kainate-treated rats were used as models of human temporal lobe epilepsy in the study. Each treatment was administered 15+ days post-implantation. Immediately prior to the kainate treatment, baseline local field potential (LFP) recordings (bandpass filtered from 10 Hz to 500 Hz) and video were obtained using a TDT System3 recording system (Tucker-Davis Technologies, Alachua, Fla., USA) and Quickcam camera (Logitech, Fremont, Calif.). In brief, each implanted rat was intra-peritoneally injected with 5 mg kg⁻¹ kainic acid, e.g., Ascent™ Scientific, Princeton, N.J., USA, hourly, until it reached a state of convulsive status epilepticus. LFP recordings were obtained along with video footage between each of four to six administered injections. Seizures were then marked out by visual inspection of data and corresponding video. A team of neurologists then verified the identified seizure patterns and marked onset times.

Each implanted animal underwent treatment of kainic acid as per the protocol until a convulsive state of status epilepticus was attained. A total of 125 seizures were marked out from six treated animals. This included both subclinical and clinical seizures scored on a racine scale of 1 through 5. Seizure onset was identified by visual inspection of electrographic and video records by a trained epileptologist. This was supplemented by marking out the first point at which the electrographic spiking activity exhibited a sharp increase in instantaneous energy with a period of gradual amplitude increase. Marked out seizures had an average duration of 65 s with a standard deviation of 27 s. In this study, no sleep-wake cycle experiments were performed, and all data used were obtained during the course of progression of the animal into chemically induced status epilepticus. As a result, there were no long interictal periods.

The algorithm described hereinabove does not assume any morphology or specific seizure pattern, making it more generic and widely applicable to the electrographic data that exhibited a combination of features including increased amplitude, sustained spiking and high-frequency activity. A set of 3-5 seizures and about 15 min of baseline activity was used to extract the thresholds for each animal in the study. Typical data segment lengths varied from 10 to 25 min. Longer continuous data intervals were used to quantify false positive rates with 20-45 min baseline recordings for each animal.

The efficacy of the algorithm described hereinabove is in part dependent on the choice of the parameters (K, IEI threshold, and count threshold) used to detect increase in amplitude, frequency and rhythmicity in the seizure phase. Hence, optimal choice of these parameters assumes paramount importance in order to co-optimize the algorithm and hardware in terms of detection latency, power consumption and number of false positives (FP) and false negatives (FN). As most algorithms need patient specific programming, a general framework is essential to optimize this process, reporting its trade-offs at each step.

The process of setting the parameters for the algorithm described hereinabove, based on statistical analysis of neural recordings, is described below. The parameter setting process is based on the distributions of IEI obtained for different values of K.

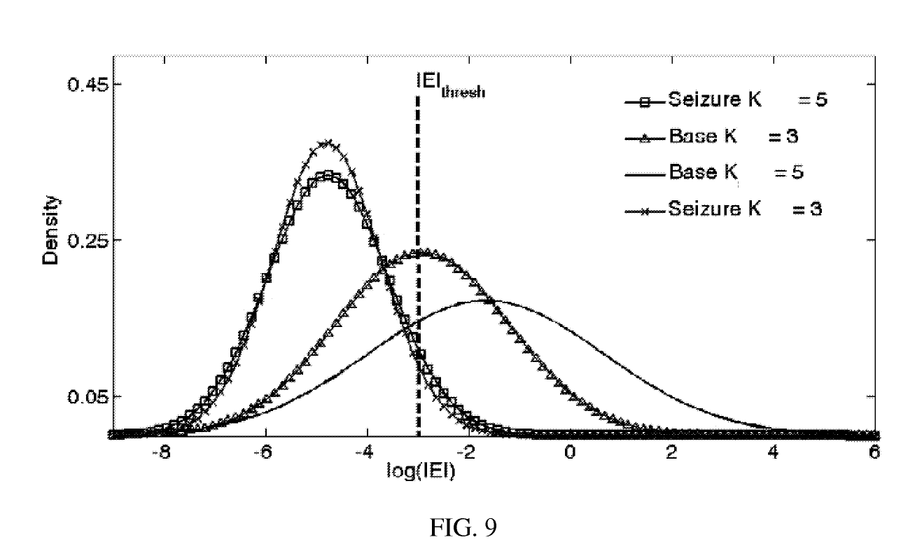

As discussed above, K times the average baseline amplitude is used as the amplitude threshold for the classification of events. Once the events are marked, intervals between pairs of successive events are calculated to obtain the distribution for IEI corresponding to the chosen K. For this analysis, data corresponding to the baseline and seizure phases are considered separately and two IEI distributions are obtained. The distribution of IEI data points is dependent in part on the choice of K. Choosing a value of K changes the number of events identified in each state, and also the intervals between them. The IEI resulting from a particular K selection resulted in a log-normal distribution, depicted in FIG. 9. The baseline distribution shows an increased standard deviation with an increase in K as fewer events are detected with a higher threshold. The seizure distribution tends to get sharper and the two distributions overlap in a narrower manner with each increase in K, potentially leading to a reduction in false positives. The depicted trend indicates that the baseline and seizure IEI distributions start to disperse away from each other with an increase in K. However, an excessively high value of K results in a high probability of entirely missing certain electrographic seizure events and/or increasing false negatives. Therefore, the choice of K should take these tradeoffs into account. Metrics of interest—detection delay, power consumption and number of false positives/negatives—are related qualitatively to cumulative distribution functions (CDFs) of IEI in the baseline and seizure phases. These tradeoffs are captured by the distributions of IEI in the baseline and seizure phases resulting from each selection of threshold.

In one implementation of the seizure detection algorithm described hereinabove, a count threshold of 9-16 was implemented. Increasing this metric was found to decrease the number of false positives in detection and marginally increase detection delay. The clinician is given the flexibility to program the hardware to the desired count threshold based on the observed false positive rate from training data as a coarse control mechanism. The optimum value for the count threshold can be determined empirically. Table 1 lists the results from the study.

TABLE 1
Hardware implementation results

| | | No. | | Avg | No. | | | | |
|---|---|---|---|---|---|---|---|---|---|
| | | Of | | Time | Of | | | | |
| ID | K | Szr | CNT | min | Det. | FP | FN | SEL | SEN |
| 1 | 5 | 18 | 4 | 22 | 16 | 2 | 2 | 0.889 | 0.889 |
| 2 | 4 | 22 | 5 | 18 | 21 | 3 | 1 | 0.875 | 0.955 |
| 3 | 6 | 15 | 8 | 16 | 14 | 2 | 1 | 0.875 | 0.934 |
| 4 | 4 | 18 | 8 | 20 | 17 | 1 | 1 | 0.944 | 0.944 |
| 5 | 4 | 17 | 6 | 12 | 17 | 3 | 0 | 0.805 | 1.000 |
| 6 | 5 | 19 | 12 | 13 | 19 | 2 | 0 | 0.904 | 1.000 |

A small subset of each animal's data was used to generate specific thresholds and these were tested using untrained data from the same animal. All results reported in table 1 were obtained using the simulated real-time environment from the segments of continuous data that excluded the training seizures and baseline. The data were segmented and longer continuous segments were used to test FP rate on the baseline data. The average duration of the continuous segments used is also reported in the table. The algorithm performed with an average sensitivity (SEN) and selectivity (SEL) of 95.3% with 95% confidence intervals [90.8%, 99.9%] and 88.9% with 95% confidence intervals [85.5%, 92.4%], respectively. Selectivity is defined as the ratio of the number of correct detections to the total number of detections and false positives, indicating a measure of rejection of false positives. Sensitivity was calculated as the ratio of number of correct detections to the total number of detections and false negatives, indicating a measure of the algorithm to detect seizure activity from baseline.

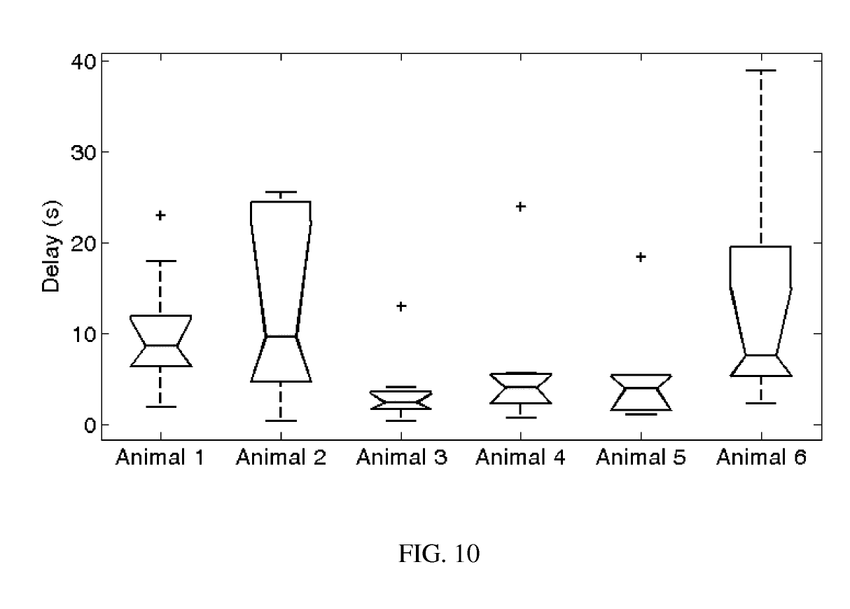

A parameter of interest in deciding the feasibility of the algorithm described hereinabove in a closed-loop system is detection delay. Detection delay is defined as the time interval between electrographic onset of the seizure and when the algorithm triggers a detection. A false negative is defined as any detection with a delay greater than half the duration of the electrographic seizure. In other words, if a detection is not made within 50% of the seizure duration (within the tonic part), it is considered a miss. FIG. 10 shows the measured detection delays from each of the implanted animals along with their median values. The algorithm described hereinabove had an overall average detection delay of 8.5 s [5.97, 11.04] with a standard deviation of 6.85 s. The large standard deviation was due to differences in a clear definition of electrographic onset and also due to animal to animal variations in the progression of a seizure event. The average detection delay provides sufficient time to alert the subject to brace for the approaching seizure as well as downstream circuits to provide measures to prevent the seizure from further progressing.

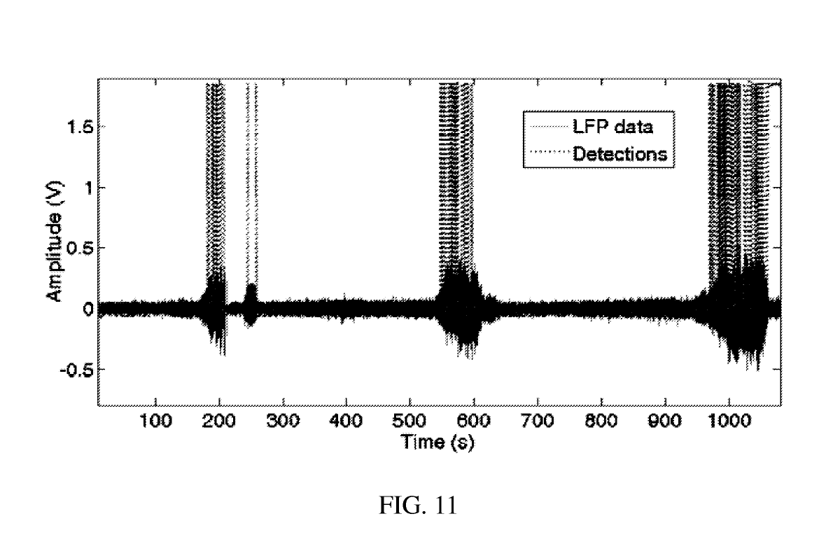

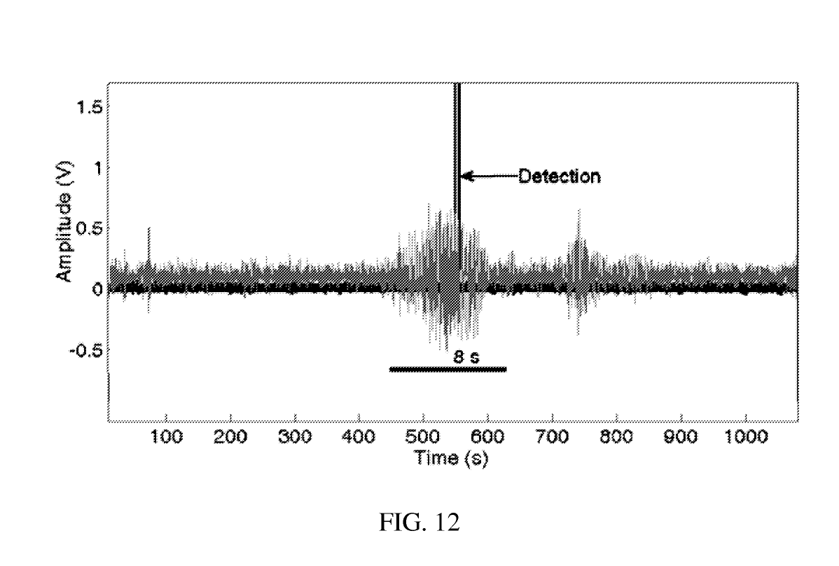

FIG. 11 depicts a graph of progression of three seizures. Seizures are detected with no false positives or misses by the hardware. In certain cases, inter-ictal bursts of high-amplitude high-frequency activity were detected by the hardware as shown in FIG. 12. Activity that was sustained for periods over 5 s and consisted of high-frequency rhythmic patterns was detected by the hardware. For the purposes of this classification, such detections were logged as false positives. It is to be noted that the false positive rate has a characteristic trade-off with detection delay as they have conflicting requirements.

The system architecture and algorithm described herein can also be combined with any custom circuit implementation of a multi-channel neural recording device at almost no additional cost of power. Such a device would enable researchers to accurately identify and track the path of a seizure away from its epileptic focus, in turn equipping them with answers to the questions surrounding when and where to stimulate. Multi-channel neural recording systems equipped with efficient detection schemes reduce the data bandwidth load on transmission schemes from these systems by only transmitting detections as opposed to digitized neural data. This would aid in long-term studies to understand the temporal dynamics of a seizure event to increase the temporal selectivity of intervention.

While the invention has been illustrated and described in detail in the drawings and foregoing description, the same should be considered as illustrative and not restrictive in character. It is understood that only the preferred embodiments have been presented and that all changes, modifications and further applications that come within the spirit of the invention are desired to be protected.

1. A method for detecting an approaching seizure, comprising:
receiving an analog neurosignal from an electrode;
comparing the analog neurosignal with a predetermined amplitude threshold to generate a plurality of digital transitions corresponding to the analog neurosignal transitioning from a first level below the predetermined amplitude threshold to a second level above the predetermined amplitude threshold;
generating a plurality of comparator signals, each comparator signal of the plurality of comparator signals corresponding to a comparison of a length of time between a pair of digital transitions of the plurality of digital transitions and a predetermined temporal threshold; and
generating a seizure detection signal if at least two of the comparator signals of the plurality of comparator signals are enabled.

2. The method of claim 1, further comprising:
separating the analog neurosignal to a first signal having a first frequency range and to a second signal having a second frequency range.

3. The method of claim 2, wherein the first signal includes frequency components from 0 to 10 Hz.

4. The method of claim 2, wherein the second signal includes frequency components from 10 to 500 Hz.

5. The method of claim 2, further comprising:
averaging the amplitude of the first signal; and
updating the predetermined amplitude threshold based on the averaged amplitude of the first signal.

6. The method of claim 2, wherein the predetermined amplitude threshold is a predetermined multiple of the averaged amplitude of the first signal.

7. The method of claim 1, further comprising:
synchronizing the plurality of digital transitions to a clock.

8. The method of claim 7, further comprising:
counting a number of clock cycles between a pair of synchronized digital transitions to generate a count of a length of time between the pair of synchronized digital transitions; and
latching the count to generate an input to a comparator to compare the latched count with a predetermined count corresponding to the predetermined temporal threshold to generate a comparator output signal of the plurality of comparator output signals.

9. The method of claim 1, wherein the predetermined amplitude threshold and the predetermined temporal threshold are adjusted according to an accumulated history of false positives and false negatives.

10. The method of claim 9, wherein the number of comparator signals of the plurality of comparator signals being enabled causing generation of the seizure detection signal is adjusted according to an accumulated history of false positives and false negatives.

11. A seizure detection system for detecting an approaching seizure, comprising:
a signal amplitude comparator configured to compare an analog neurosignal and a predetermined amplitude threshold and generate a plurality of digital transitions corresponding to the analog neurosignal transitioning from a first level below a predetermined amplitude threshold to a second level above the predetermined amplitude threshold; and
at least one comparator generating a plurality of comparator signals corresponding to a comparison of a length of time between a pair of digital transitions of the plurality of digital transitions and a predetermined temporal threshold, wherein a seizure detection signal is generated if at least two of the comparator signals of the plurality of comparator signals are enabled.

12. The seizure detection system of claim 11, further comprising:
a low pass filter configured to separate the analog neurosignal to a first signal having a first frequency range and to a second signal having a second frequency range.

13. The seizure detection system of claim 12, wherein the first signal includes components from 0 to below 10 Hz.

14. The seizure detection system of claim 12, wherein the second signal includes components from 10 to 500 Hz.

15. The seizure detection system of claim 12, further comprising:
a moving average block configured to average the amplitude of the first signal; and
an updating circuit configured to update the predetermined amplitude threshold based on the averaged amplitude of the first signal.

16. The seizure detection system of claim 15, wherein the predetermined amplitude threshold is a predetermined multiple of the averaged amplitude of the first signal.

17. The seizure detection system of claim 12, further comprising:
a synchronization circuit configured to synchronize the plurality of digital transitions to a clock.

18. The seizure detection system of claim 17, further comprising:
a counter configured to count a number of clock cycles between a pair of synchronized digital transitions to generate a count of a length of time between the pair of synchronized digital transitions; and
a latch configured to latch the count to generate an input to a comparator of the plurality of comparators to compare the latched count with a predetermined count corresponding to the predetermined temporal threshold to generate a digital comparator output.

19. The seizure detection system of claim 11, wherein the predetermined temporal threshold is adjusted according to an accumulated history of false positives and false negatives.

20. The seizure detection system of claim 19, wherein the number of comparator signals of the plurality of comparator signals being enabled causing generation of the seizure detection signal is adjusted according to an accumulated history of false positives and false negatives.
